(19) United States
(12) Patent Application Publication
(10) Pub. No.: US 2022/0047879 A1
(43) Pub. Date: Feb. 17, 2022

(54) SYSTEM AND METHOD FOR DELIVERY A FLOW OF ELECTRONS FOR INHALATION OR INGESTION BY AN ORGANISM

(71) Applicant: Zhen Li, Toronto (CA)

(72) Inventor: Zhen Li, Toronto (CA)

(21) Appl. No.: 16/994,598

(22) Filed: Aug. 16, 2020

(51) Int. Cl. A61N 1/44; A61M 16/00; H02M 1/10; H02M 3/335; H02M 7/217

(52) U.S. Cl. CPC A61N 1/44; A61M 16/0003; H02M 2001/0009; H02M 3/33523; H02M 7/2176; H02M 1/10

(57) ABSTRACT

A system is disclosed for supplying a system and method for delivering a flow of electrons to an organism's airway for inhalation or mouth for ingestion. The system includes a pre-stage rectification and filtering module, a power conversion module, a post-stage rectification and filtering module, an output module, an over voltage protection module, a current-limiting protection module, and a pulse width controller. Each of these modules and components is configured to deliver a suitable voltage and flow of electrons via the output module comprising a negative electrode that emits electrons into the air stream, which then enters the organism's air passages (e.g. nose). Electrons emitted through the skin of the organism are captured by the output module's positive electrode. The flow of electrons from the system, to and through the organism and back to the system promotes cell metabolism and improved biological functioning of the organism.

### FIELD OF THE INVENTION

The present invention relates to the field of biotechnology, more specifically to a system and method for delivering a flow of electrons to an organism's airway for inhalation or mouth for ingestion.

### BACKGROUND OF THE INVENTION

The background to the invention provides information about the state of the art relating to the application of electron flow to promote well-being in organisms.

Electronics participate in the activities of the nervous system in the nervous system, giving the nervous system vitality and a basic material basis; electrons promote the metabolism of cells and improve the function of biological systems. If enough electrons flow through an organism this is conducive to the survival and evolution of living things.

Although there are some instruments known in the art for supplying negative ions or negative oxygen ions to an organism, such systems tend to only define the use of a positive or negative electrode, but not both. As a result, the flow of electron movement (amount and direction) cannot be effectively monitored and controlled. For example, CN207461460 only applies a negative electrode, but does not provide a means for controlling the flow of electrons through and out of the organism's skin back into the disclosed (device) system. Since there is no guided path or cycle loop for the electrons to flow along it is not possible to know where the negative ions go, reducing the effectiveness for the organism in terms of controlling the further supply of electrons to the body. In CN1309889A, only a positive electrode is used. Without positioning a negative electrode, it is unclear where electrons flowing into the organism come from and again more difficult to control the levels received by the organism.

Other systems known in the art that deliver negative ions via a subject's respiratory track (airway) are also deficient in terms of their control over the path of the flow of electrons in and out of the subject's body. For example, in CN108904937 a system is disclosed for purifying air and then generating negative ions from the purified air. Beyond this, however, there is no disclosure or teaching regarding the configuration of the system to provide for path of electron flow into and out of the body an organism and back into the system.

In CN205145323, similar to CN108904937, one of the technical problems being solved is the production of a sufficient concentration of negative anions for uptake through the respiratory system of an organism and the solution provided is an apparatus for generating sufficient anions for delivery to a subject and not to provide for path of electron flow into and out of the body an organism back into the system.

Other systems known in the art include the disclosure of CN107744621 where anions are generated using a chemically mediated process and delivered to a subject via a mask, however, the reliance on chemical processes creates added levels of system complexity (for example in the configuration of the respiratory mask), maintenance and risks for the subject or user of the system.

In yet another system known in the art, WO 80/00918 provides a means for a subject to breathe air charged with negative ions, while at the same time being subjected to a unidirectional electrostatic field created between a negative potential plate and filed points directed towards the subject's head and a positive potential plate in contact with the subject's body (at their feet). This results in a flow of electrons from the head to the feet of the subject. The system design more particularly comprises an inhalation cabin provided at its lower part with a conductive floor connected to the earth and at its upper part with a ceiling lamp constituted by a conductive plate furnished with a multiplicity of peaks, a static electricity generator whose negative pole is connected to said ceiling lamp and the positive pole to ground, and a negative ion generator comprising an input supplied with pressurized air, an outlet connected to the inhalation cabin, a conductive grid furnished with a multiplicity of points directed towards the outlet and a second static electricity generator, the negative pole of which is connected to said grid and the positive pole to ground. The complexity of this system in part arises from the dual methods sought to be applied and renders the apparatus impractical for use by a subject anywhere they may be, with or without the aid of a system operator. The delivery path and direction of electron flow is also inefficient and not optimal in this system. The flow of electrons begins from the cracking machine and follows a path to the diffuse air environment in the chamber where the organism is located. The electrons enter the skin of the organism and airways and then come out from the feet of the organism where the positive electrode is positioned within the chamber. Furthermore, the disclosed electrostatic machine creates electromagnetic interference which can reduce the effectiveness of the flow of electrons through the subject. The Static electricity generator WO 80/00918 depends on the turning of the motor (39) which rub two kind of material to create static electricity, thus will create a lot electromagnetic interference.

A need still remains for an effective level of electron/anion delivery to a subject through an airway or the mouth that is simple to use, controllable to meet the needs of a subject and amenable to configurations suited for independent use by the subject and portability.

### SUMMARY OF THE INVENTION

The present invention relates generally to a system for supplying a flow of electrons into an air stream being inhaled, or substance being ingested by an organism.

The system comprises a power supply, a pre-stage rectification and filtering module, a power conversion module, a post-stage rectification and filtering module, an output module, an over voltage protection module, a current-limiting protection module (to no more than 10 mA), and a pulse width controller. Each of these modules and components is configured to deliver suitable voltages and a flow of electrons via the output module that further comprises negative and positive electrodes. When the system is operatively coupled to a breathing device, the flow of electrons is emitted from a negative electrode into the air stream delivered by the breathing device, enters the organism's air passages (e.g. through the mucosa of the nose) and then exits the organism through the skin of the organism's entire body. The positive electrode may be positioned to contact the organism, directly or indirectly, and captures electrons emitted from the skin, resulting in a cycling of electrons into and through the organism and back to the system. In this manner, the output voltages of the system connect to a positively charged surface(s) around the organism to create an even electric field around the body of the organism, and the formation of an electrical circuit including the system and organism. The application of the system using a method according to the present disclosure promotes cell metabolism and improved biological functioning of the organism.

An object of the present invention is to operably couple to a breathing device a system for supplying a flow of electrons into an air stream delivered to an organism's airways by the breathing device. The system of the present disclosure may be integrated within the breathing device, or may be configured as a separate apparatus connected to the breathing device. It is another object of the present invention to allow for the continued supply of a flow of electrons when an organism is ingesting food or drink from the mouth.

In one aspect there is provided a system for supplying a flow of electrons for inhalation or ingestion by an organism, comprising:
an AC or DC power source for providing an input voltage to the system;
a pre-stage rectification and filtering module for transferring the input voltage to a power conversion module for the conversion of the input voltage into a system voltage that can power the system;
a post-stage rectification and filtering module for receiving a portion of the system voltage from the power conversion module and generating one or more output voltages, comprising one or more capacitors for mitigating the effect of electro-magnetic interference on the generation of the one or more output voltages and flow of electrons;
an output module for receiving the one or more output voltages from the post-stage rectification and filtering module, the output module comprising a negative electrode and positive electrode for, respectively, emitting the flow of electrons and receiving a flow of electrons from the skin of the organism, wherein the positive electrode has a surface area sufficient to receive said flow of electrons from substantially all of the skin of the organism;
an over voltage protection module connected to the output module for monitoring and limiting the amount of the one or more output voltages;
a current-limiting protection module connected to the output module for monitoring and limiting the amount of current running through the system; and
a pulse width controller connected to the output module, over voltage protection module, current limit protection module and power conversion module for controlling the one or more output voltages and flow of electrons emitted from the negative electrode;
   wherein the negative electrode is integrated into a conduit of the air delivery device to supply the flow of electrons to the air stream as the air stream passes through the conduit and is delivered to the organism or to a substance being ingested by the organism, and wherein the positive electrode is configured around the body of the organism to pull electrons emitted from substantially all of the skin of the organism back into the system.

In one embodiment, when the flow of electrons supplied by the negative electrode comes into contact with the air stream, anions are generated and carried by the air stream to the organism or substance being ingested by the organism.

In another embodiment, the anions release electrons into the body of the organism or substance being ingested by the organism, causing electrons to move through the body of the organism.

In a further embodiment, the positive electrode is in contact with the skin of the organism.

In still another embodiment, a non-conductive woven, net-like or mesh material separates the skin of the organism from the positive electrode it is in contact with.

In yet another embodiment, the conduit is a flexible hose.

In still a further embodiment, the negative electrode comprises one or more layers of conductive mesh inserted into the conduit to supply the flow of electrons emitted from the negative electrode to the air stream or substance being ingested by the organism.

In yet a further embodiment, the one or more layers of conductive mesh is housed in an assembly incorporated into the conduit and configured such that the air stream passes through the one or more layers of conductive mesh to come into contact with the flow of electrons emitted from the negative electrode.

In still a further embodiment, the one or more layers of conductive mesh form a cylinder of material with about a diameter of 50 mm and about a depth of 20 mm.

In yet another embodiment, the positive electrode for receiving electrons emitted from the organism comprises one or more segments.

In yet a further embodiment, the one or more segments are first and second plates each with a surface area spanning the entire length and width of the organism, wherein the first plate is positioned behind or under the back of the organism and the second plate is positioned in front or over top of the organism when the system is in use.

In yet a further embodiment, a wire connects the negative electrode to the system and provides a source of electrons for the flow of electrons emitted from the negative electrode.

In another embodiment, wherein the wire can be disconnected from the negative electrode and connected to a conductive lining in a container to provide a flow of electrons to the substance being ingested by the organism.

In another aspect there is provided a method for supplying a flow of electrons for inhalation or ingestion by an organism using a system comprising:
an AC or DC power source for providing an input voltage to the system;
a pre-stage rectification and filtering module for transferring the input voltage to a power conversion module for the conversion of the input voltage into a system voltage that can power the system;
a post-stage rectification and filtering module for receiving a portion of the system voltage from the power conversion module and generating one or more output voltages, comprising one or more capacitors for mitigating the effect of electro-magnetic interference on the generation of the one or more output voltages and flow of electrons;
an output module for receiving the one or more output voltages from the post-stage rectification and filtering module, the output module comprising a negative electrode and positive electrode for, respectively, emitting the flow of electrons and receiving a flow of electrons from the skin of the organism, wherein the positive electrode has a surface area sufficient to receive said flow of electrons from substantially all of the skin of the organism;
an over voltage protection module connected to the output module for monitoring and limiting the amount of the one or more output voltages;
a current-limiting protection module connected to the output module for monitoring and limiting the amount of current running through the system; and
a pulse width controller connected to the output module, over voltage protection module, current limit protection module and power conversion module for controlling the one or more output voltages and flow of electrons emitted from the negative electrode;
   wherein the negative electrode is integrated into a conduit of the air delivery device to supply the flow of electrons to the air stream as the air stream passes through the conduit and is delivered to the organism or to a substance being ingested by the organism, and wherein the positive electrode is configured around the body of the organism to pull electrons emitted from substantially all of the skin of the organism back into the system.

In another aspect there is provided a use of a system for supplying a flow of electrons for inhalation or ingestion by an organism, the system comprising:
an AC or DC power source for providing an input voltage to the system;
a pre-stage rectification and filtering module for transferring the input voltage to a power conversion module for the conversion of the input voltage into a system voltage that can power the system;
a post-stage rectification and filtering module for receiving a portion of the system voltage from the power conversion module and generating one or more output voltages, comprising one or more capacitors for mitigating the effect of electro-magnetic interference on the generation of the one or more output voltages and flow of electrons;
an output module for receiving the one or more output voltages from the post-stage rectification and filtering module, the output module comprising a negative electrode and positive electrode for, respectively, emitting the flow of electrons and receiving a flow of electrons from the skin of the organism, wherein the positive electrode has a surface area sufficient to receive said flow of electrons from substantially all of the skin of the organism;
an over voltage protection module connected to the output module for monitoring and limiting the amount of the one or more output voltages;
a current-limiting protection module connected to the output module for monitoring and limiting the amount of current running through the system; and a pulse width controller connected to the output module, over voltage protection module, current limit protection module and power conversion module for controlling the one or more output voltages and flow of electrons emitted from the negative electrode;
   wherein the negative electrode is integrated into a conduit of the air delivery device to supply the flow of electrons to the air stream as the air stream passes through the conduit and is delivered to the organism or to a substance being ingested by the organism, and wherein the positive electrode is configured around the body of the organism to pull electrons emitted from substantially all of the skin of the organism back into the system.

In certain embodiments, a flow of electrons moves through the body of the organism to revive the organism from a reduced metabolic state.

In other embodiments, the organism is suffering from a respiratory condition selected from asthma, bronchitis, pertussis, pneumonia, COVID-19, fluid in the lungs, Emphysema, or damaged lung tissue and the flow of electrons through the organism is used to ameliorate the respiratory condition.

In still another embodiment, the organism is suffering from a gastrointestinal condition selected from gastroesophageal reflux (e.g. GERD or acid reflux), gall stones, Celiac Disease, Crohn's Disease, Ulcerative Colitis, Irritable Bowel Syndrome, Diverticulitis, Hemorrhoids, and anal fissures and the flow of electrons through the organism is used to ameliorate the gastrointestinal condition.

### BRIEF DESCRIPTION OF THE DRAWINGS

These and other features of the invention will become more apparent in the following detailed description in which reference is made to the appended drawings.

FIG. 1: is a schematic diagram of a connection between a breathing device incorporating a system according to the present disclosure including the integration of a conductive mesh in a conduit for the delivery of an air stream to a mask worn by an organism.

FIG. 2: is a schematic diagram of an embodiment of the configuration of a system, and breathing device within an environment for the delivery of a flow of electrons to a subject according to the present disclosure.

FIG. 3: is a cross-sectional view of a conduit of a breathing device incorporating a conductive mesh.

FIG. 4: is a circuit block diagram of an embodiment of a system according to the present disclosure.

FIG. 5: is an embodiment of a circuit schematic according to the present disclosure for the generation of a flow of electrons according to the present disclosure.

FIG. 6 (A-D): illustrates various views of a conductive mesh assembly shown in a transparent configuration to depict the conductive mesh internal to the assembly. The assembly can be integrated as part of a conduit (e.g. flexible hose) of a breathing device and the various views include a side view (D), top view (B), isometric top view (C) in between the side and top views and an isometric side view (A) to the left of the isometric top view. The hole through the assembly as seen in the top view (B) illustrates how the conductive mesh is positioned to span the hole perpendicular the direction flow of an air stream through the conduit.

FIG. 7: illustrates schematically a wrap-around positive electrode conductive surface, which could be a rigidly constructed tube structure or pod-like structure. Alternatively, the wrap around conductive surface could be a flexible conductive material that wraps around the body of an organism.

FIG. 8 (A and B): illustrates alternative ways of delivering electrons when an organism is drinking and eating instead of receiving an air stream for inhalation according to the present disclosure, either using an air stream passing through a conductive mesh (A) into the substance being ingested, or using a (negatively charged) conductive lining in a container holding the substance being ingested by the organism (B).

### DETAILED DESCRIPTION OF THE INVENTION

Unless defined otherwise, all technical and scientific terms used herein have the same meaning as commonly understood by one of ordinary skill in the art to which this invention pertains.

The use of the word “a” or “an” when used herein in conjunction with the term “comprising” may mean “one,” but it is also consistent with the meaning of “one or more,” “at least one” and “one or more than one.” Where only one item is intended the term “one” or similar language is used.

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, device, article, system, use or method, denotes that additional elements and/or method steps may be present, but that these additions do not materially affect the manner in which the recited component, device, apparatus, system, use or method functions. The term “consisting of” when used herein in connection with a component, device, apparatus, system, use or method, excludes the presence of additional elements and/or method steps. A component, device, apparatus, system, use or method described herein as comprising certain elements and/or steps may also, in certain embodiments consist essentially of those elements and/or steps, and in other embodiments consist of those elements and/or steps, whether or not these embodiments are specifically referred to.

As used herein, the term “about” refers to an approximately +/−10% variation from a given value. It is to be understood that such a variation is always included in any given value provided herein, whether or not it is specifically referred to.

The recitation of ranges herein is intended to convey both the ranges and individual values falling within the ranges, to the same place value as the numerals used to denote the range, unless otherwise indicated herein.

The use of any examples or exemplary language, e.g. “such as”, “exemplary embodiment”, “illustrative embodiment” and “for example” is intended to illustrate or denote aspects, embodiments, variations, elements or features relating to the invention and not intended to limit the scope of the invention.

As used herein, the terms “connect” and related derivatives refer to any direct or indirect physical association between elements or features of the system, apparatus and/or devices of the present disclosure. Accordingly, these terms may be understood to denote elements or features that are partly or completely contained within one another, attached, coupled, contacting, integrated, incorporated, disposed on, joined together, etc., even if there are other elements or features intervening between the elements or features described as being connected. In the case of describing the flow of electrons according to the present disclosure, where there are intervening elements or features (e.g. the air, atmosphere or non-conductive material) between an electrode and a subject, contact between a conductive surface and a subject (organism or substance being ingested by the organism) means direct or indirect contact (e.g. separated by an organic mesh material) with the skin or surface of a subject. It is noted, however, that direct contact between a conductive surface (of a positive electrode) and the skin of a subject is also possible. It is also noted that the positive electrode can function at a distance from the skin of the organism or substance being ingested by the organism and pull electrons into the system, so long as a suitable field is created to draw the emitted electrons to the positive electrode(s) into the system.

The terms “organism” of “subject” as used herein refers to a human or non-human animal, which may include mammals and other air breathing animals which are cared for by humans in different contexts. An aquatic organism or subject may also benefit from the application of the system and method of the present disclosure with suitable modifications that one skilled in the art could produce so as to achieve the functionalities and objects of the present disclosure. A subject may be inhaling or ingesting a flow of electrons supplied by the system according to the present disclosure. When ingesting electrons, the flow of electrons is delivered to a substance being ingested by an organism, including soft and hard foods (e.g. breads, fruit, vegetables, stews, soups, porridge, meats, fish, cheeses), and a variety of drinks and other edible products.

The terms “therapy” and “treatment,” as used interchangeably herein, refer to an intervention performed with the intention of promoting good health and general wellness, as well as alleviating the symptoms associated with, preventing the development of, or altering the pathology of a disease, disorder or condition. Thus, the terms therapy and treatment are used in the broadest sense, and in various embodiments include one or more of the prevention (prophylaxis), moderation, reduction, amelioration and/or curing of a disease, disorder or condition at various stages. Subjects in need of therapy/treatment thus may include those already having the disease, disorder or condition as well as those prone to, or at risk of developing, the disease, disorder or condition and those in whom the disease, disorder or condition is to be prevented.

It is contemplated that any embodiment of the compositions, devices, articles, methods and uses disclosed herein can be implemented by one skilled in the art, as is, or by making such variations or equivalents without departing from the scope and spirit of the invention.

System and Method for Supplying Electrons to an Air Stream

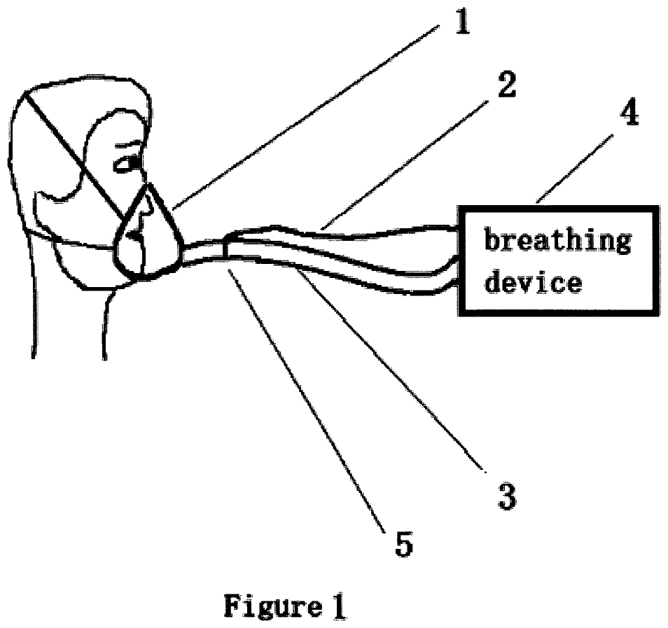

The technical solutions in the embodiments of the present invention will be clearly and completely described with reference to the attached drawings, FIGS. 1-7. The system of the present disclosure can be understood to comprise three key functional aspects, an electron flow generating aspect, an electron supply aspect and an electron flow control aspect. FIG. 1 illustrates the electron flow generating aspect and electron supply aspect. A breathing device 4 incorporating a system according to the present disclosure is provided and further comprises a conduit 3 for the delivery of an air stream to an organism via a mask 1. One skilled in the art would appreciate that it is important to ensure the air stream delivered by the breathing device is of a good quality and can apply a number of technologies to ensure the air is clean or purified of unwanted elements prior to reaching the conductive mesh 5 of the present disclosure. A flow of electrons is generated by the system from inside the breathing device 4 and provided via the wire 2 to the negative electrode comprising conductive mesh 5. The conductive mesh 5 supplies the flow of electrons to the air stream moving through the conduit 3 at a selected location as close as possible to the mask 1 where it is taken in by the organism (subject) through its air passages, e.g. the nose. The positioning of the conductive mesh 5 within the conduit 3 will be influenced by the need to ensure the anions created reach the air passages of the organism and the safety of the organism, or health care provider when tending to the organism, against unintentional touching of the negative electrode.

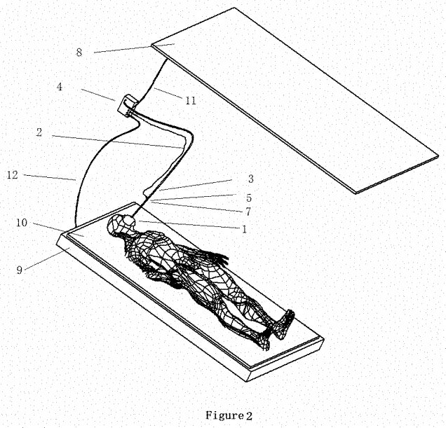

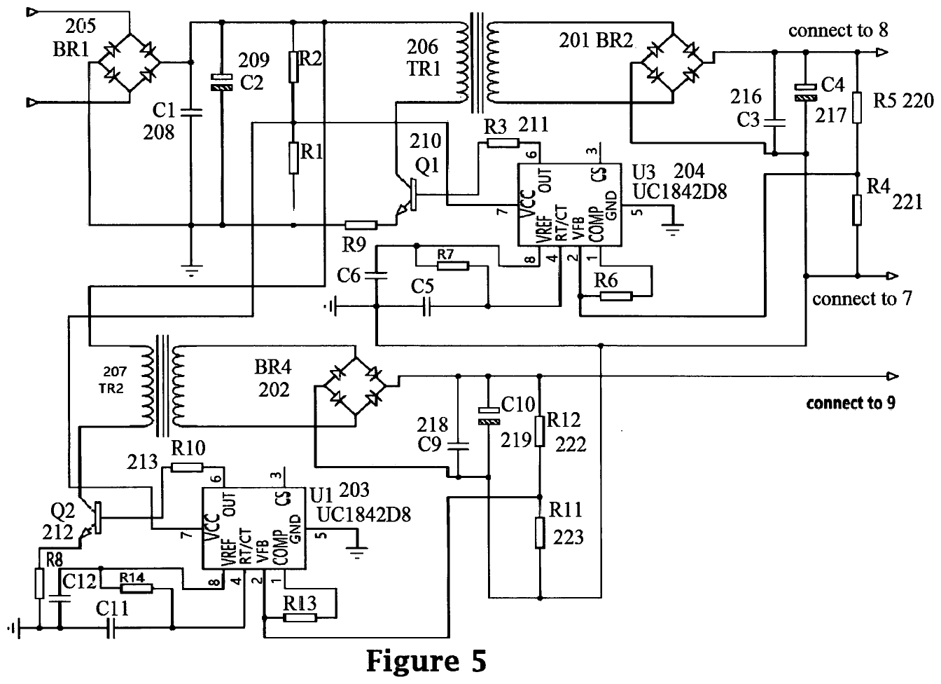

FIG. 2 illustrates the full system integrated with the breathing device including the electron control aspect. The breathing device 4 houses the electronic circuit components of the system of the present disclosure (as further exemplified in FIG. 5) and provides an air stream to the organism for inhalation, and which may or may not be enriched in oxygen content. In addition to the conduit 3 connected to the breathing device 4, a wire 2 connected to the conductive mesh 5 at connection point 7 for supplying a continuous flow of electrons to the air stream passing through conduit 3. Wires 11 and 12 connect the system to two positive electrode segments 8 and 9 (in the form of conductive plates), respectively. The positive electrode segments 8, 9 are large enough to span the area taken up by an organism and the organism is in contact with positive electrode segment 9, lying down on the positive electrode segment 9 on their back (or could be lying down on their abdomen in the alternative). A layer of non-conductive mesh material 10 separates the skin of the organism from the conductive surface of the positive electrode 9. The distance of the positive electrode segments 8, 9 form the body of the organism can be adjusted to accommodate the needs of organism in a manner so as to be able to, on a continuous, uninterrupted basis, capture or receive electrons emitted or emanating from all of the skin (hairs) of the organism and cycle said electrons back to the system's electronic component circuitry incorporated into the breathing device 4.

When using two or more positive electrode segments conductive material segments, versatility is provided to health care providers to move segments as needed to attend to the different needs of the organism while still maintaining some flow of electrons through the system and organism. If segments are moved to a defined location, the system voltage can be adjusted temporarily to account for this change in segment positioning relative to the organism. When the health care provider no longer needs to attend to the organism, the segment that has been moved can be returned to its normal location and the output voltage adjusted accordingly for the desired functioning of the system and flow of electrons.

Accordingly, the organism must be sufficiently close to the positive electrode surface(s) to complete the electronic circuit created by the system and organism and provide an accurate reading regarding the returning flow of electrons, so that the system can make adjustments to the output voltage(s) using a pulse width controller (which may comprise a microcontroller) to control the flow of electrons out of the negative electrode/conductive mesh 5 and back to the system.

The placement distance between a positive electrode conductive material and subject's skin, can be changed and adjusted when needed by changing the voltage of a plate embodiment of the positive electrode. Accordingly, the design of the positive electrode conductive material can be selected by one skilled in the art to be manipulated to provide, as much as practicable, a substantially defined distance between the positive electrode conductive material and majority of the skin surface of the subject's body. In doing so, one skilled in the art can then also make appropriate adjustments in output voltage to facilitate the flow of electrons without a break in the electrical circuit formed with the system of the present disclosure and the subject.

In one embodiment, the positive electrode conductive material may be configured to allow for the manipulation of segments of material. The positive electrode may be made of a continuous piece of material, segments of which can be manipulated or moved relative to other segments (e.g. in the case of a blanket or sleeping bag to operate as two or more positive electrode segments. The positive electrode may also be made of several distinct pieces of conductive material to form or operate as two or more positive electrode segments.

The distance range between the organism and positive electrode segments will dictate the voltage outputs by the system. In general, the closer the positive electrode (or segment thereof) is to the skin of the organism, the more efficiently the system works to receive a flow of electrons from the organism. A voltage output range of no more than about 500V will be used when the positive electrode is in contact with the body of the organism. A voltage output range of no more than about 80,000V will be used when the positive electrode (or segment thereof) is not contacting the body of the organism, e.g. from several inches to several feet away from the organism (e.g. up towards the ceiling of a room, or far enough from the organism to allow the organism to eat, move, or interact with other individuals. By way of further example, health providers or home care givers can attend to the varied needs of a subject while the system remains in uninterrupted use by configuring the positive electrode to have segments that can be pulled away from or raised above the body of the organism for these individuals to attend to the organisms needs. When the distance between a segment of the positive electrode and the organism is changed the voltage to that segment can be altered to ensure there is an equal field of pulling force on the electrons emitted from the skin of the organism by the positive electrode around the body of the organism.

In the embodiment of FIG. 2, the positive electrode has two segments in the form of plates each with a surface area spanning the full length and width of the body of a human subject. With the positioning of the two plates shown in FIG. 2, the output voltage for the top plate would be higher than the one in the bottom, so that the electric field strength is the same around the body, and the pulling force on the electrons released by the skin of the organism is the same for both sides of the body. This type of configuration ensures the electrons flow in a consistent direction from the organism to the system and minimizes the creation of electromagnetic interferences that can affect the flow of electrons and integrity of the electrical circuit formed by the system and organism.

A similar configuration of upper and lower positive electrodes, as well as providing positive electrode conductive surfaces for both sides of the body, can be achieved using a pod-like structure (akin to tanning salon beds). In such structures, the inner surface is lined with positive electrode materials in several segments, which allow the voltages applied to each segment to be controlled by the system relative to the distance of said segments from the body at any given inner surface point within the pod-like structure.

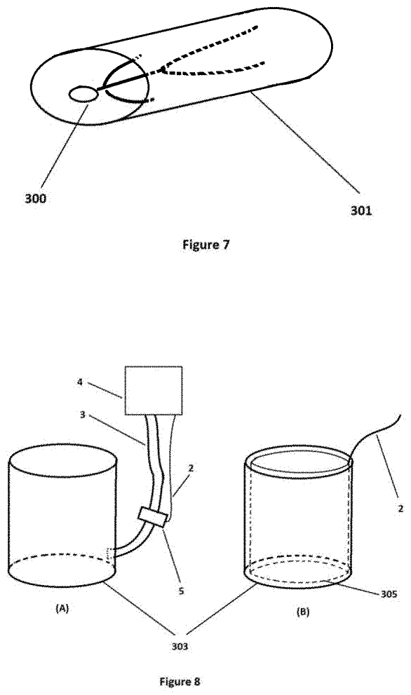

In an alternative embodiment, the positive electrode(s) may be made of flexible conductive material that can be rolled out like a mat underneath the organism, cover the organism like a blanket, or wrap around the organism 300 like a sleeping bag 301, as shown in FIG. 7, or otherwise be incorporated into apparel worn by the organism while standing or sitting (e.g. a “onesie”, pajama or jumpsuit).

In an embodiment where a single positive electrode is used, such as a unitary apparel for the body (e.g. jumpsuit, overalls, “onesies”), the voltage applied takes into account that the distance of the positive electrode conductive material from the skin of the organism is essentially the same distance over most of the surface of body. Similarly, wearing multiple apparel pieces, e.g. a shirt and pants, lined with positive electrode conductive material will also result in essentially the same distance between said positive electrode conductive material and organism's skin over most of the surface of body.

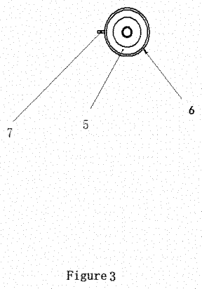

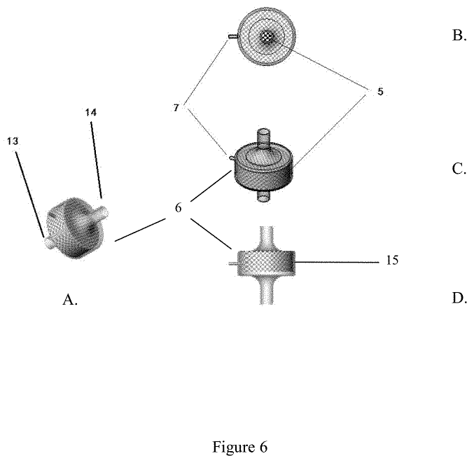

With reference to FIGS. 3 and 6 (views A-D) a means for incorporating or integrating the negative electrode/conductive mesh 5 into the conduit 3 of the breathing device 4 is provided. A conductive mesh assembly 6 is provided and comprises a chamber (or shell portion) 15 for housing one or more layers of conductive mesh 5, and two tube-like opposing extensions 13, 14 protruding from the top and bottom sides of the chamber 15 and aligned to form a hole from the end of one tube-like extension 14 through the chamber 15 to the end of the other tube-like extension 13. Each of the tube-like extensions (as shown in FIG. 6) can be inserted into tubing (e.g. flexible hose) used to form the conduit 3 of the breathing device 4. A connection point or interface 7 is provided at the side wall of the chamber 15 of the assembly 6 to attach the wire 2 shown in FIGS. 1 and 2 to the conductive mesh 5. To further illustrate the use of the conductive mesh assembly, a conduit 3 made of flexible hose with an outer diameter of, for example, about 7 mm and inner diameter of about 5 mm, can be cut with one cut end then being connected to the tube-like extension 14 and the other cut end being connected to the tube-like extension 13. The configuration of the assembly of this embodiment is also convenient to allow for its temporary removal from the conduit to allow for the replacement of conductive mesh as it gets oxidized over time and has reduced capacity to emit electrons.

In addition to the layered cylindrical conductive mesh material illustrated in FIG. 3 other conductive mesh structures and configurations can be integrated into the conduit of the breathing device.

In one embodiment, multiple conductive mesh assemblies may be inserted within the conduit of the breathing device to increase the supply of electrons to the air stream. In another embodiment, instead of providing conductive mesh in a cross-sectional orientation the internal wall forming the channel of the conduit can be lined with conductive mesh for a defined length selected by the skilled technician to provide a flow of electrons to the air stream for delivery to an organism. In yet another embodiment conductive mesh spheres may be stacked inside the conduit of the breathing machine.

The conductive mesh can be made from a number of conductive materials, such as a metal (e.g. copper, silver, gold) and semi-conductive material such as graphene (e.g. in the form of a carbon fiber mesh). The conductive mesh will have sufficient structural strength to span the cross section of the flexible hose or other conduit to be secured therein and not collapse out of the perpendicular orientation relative to the direction of the air stream flow through the conduit. Alternatively, the use of the conductive mesh assembly provides a means to provide structural support for the placement of the conductive mesh and maintain its cross-sectional orientation within the hose. The use of multiple layers of mesh also provides additional structural support and surface area from which electrons can be emitted and supplied to the air stream passing through the conduit/hose.

When air flows through the conductive mesh, the electrons are carried to the nasal mucous and mucosa, and the lungs of the organism via the mask. The amount of anions or negative ions (electrons carried by the air stream) prior to entering the air passages (e.g. near the nose) account for not less than about 1,000 electrons per cubic centimeter compared to natural environmental levels averaging in between about 200 to about 800 electrons per cubic centimeter. In one embodiment, the system delivers to an organism from about 1,000 to about 10,000 electrons per cubic centimeter. To measure the concentration of electrons per cubic centimeter, a meter device (air ion counter) can be used and incorporated into the system according to the present disclosure to provide this information to the system and be factored by the control mechanism (pulse width controller) to adjust the output voltage(s) accordingly.

The conductive mesh assembly provides a means to create a space that is filled with conductive mesh and ensure that all air of the air stream passes through the conductive mesh to provide an even distribution of electrons/anions in the air stream which then continues to the organism.

In one embodiment, the diameter of the chamber 15 of the assembly 6 (comprising the one or more layers of conductive mesh 5) is substantially the same as the diameter of the conduit 3. In another embodiment, the diameter of the chamber 15 of the assembly 6 chamber 15 comprising the one or more layers of conductive mesh 5 is larger than the diameter of conduit 3. In a further embodiment the one or more layers of conductive mesh 5 has a diameter substantially equivalent to the diameter of the inner channel formed by the conduit 3. In yet a further embodiment the one or more layers of conductive mesh 5 has a diameter that is larger than the diameter of the inner channel formed by the conduit 3. In still another embodiment, the one or more layers of conductive mesh 5 has a diameter of about 50 mm and depth (thickness) of about 20 mm.

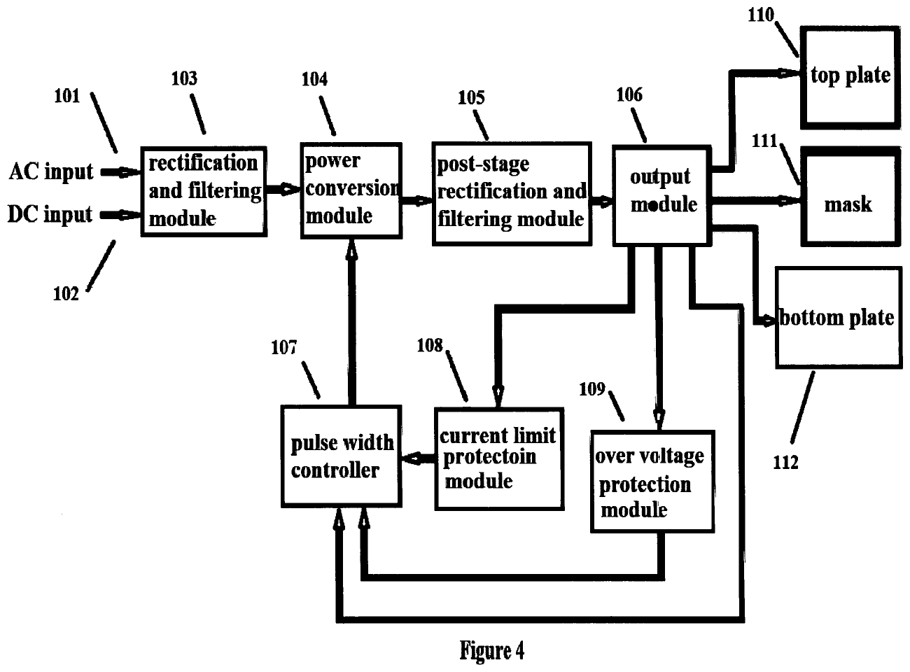

With reference to FIG. 4, in one embodiment, the system of the present disclosure comprises an AC or DC power supply to provide an AC input voltage 101 or DC input voltage 102, a pre-stage rectification and filtering module 103, a power conversion module 104, a post-stage rectification and filtering module 105, an output module 106 further comprising positive 110, 112 and negative outputs 111 to the positive and negative electrodes positioned in this embodiment at conductive material in plates (positive electrodes) and near the mask (negative electrode) of the breathing device. The system also comprises an over voltage protection module 109, a current limiting protection module 108, and a pulse width controller 107 (which may include a microcontroller). The pre-stage rectification and filtering module 103 transfers the input voltage to the power conversion module 104, the power conversion module 104 converts the input voltage to a system voltage and outputs it to the post-stage rectification and filtering module 105. The post-stage rectification and filtering module sends the output voltage(s) to the output module 106, and the output modules respectively directs the output voltage(s) to the negative electrode and the top plate and the bottom plate via outputs 110, 111 and 112. The output module 106 is also connected to the pulse width controller 107, the over voltage protection module 109 and the current limit protection module 108, while the pulse width controller 107 is also connected to the over voltage protection module 109, the power conversion module 104, and the current limit protection module 108.

A flow of electrons is injected (supplied) into the breathing device's flexible hose (conduit) leading to the air passages of the organism via the mask (e.g. through the nose), and the electrons attach to molecules in the air and turn air molecules into anions. The anions enter the air passages of the organism and reach the mucosa of said air passages and lungs of the organism where they release electrons. In this way, the anions function as carriers of the flow of electrons to the tissue of the organism to maintain a continuous flow of electrons into and through the organism.

The electrons circulate in the organism and then come out from the skin, where they are captured (received) by the positive electrode. The positive electrode forms a voltage quasi-isobaric layer around the organism (consistent electric field at a given distance from the body, at any point from around the body).

As shown in FIG. 2 in one embodiment there are two positive electrode segments in the form of plates 8 (top) and 9 (bottom). The plates provide flat surfaces that are made of conductive material (selected from materials similar to what the conductive mesh is made of), having different potentials to produce the desired electric field strength. A further non-conductive mesh structure 10 is provided to support the subject to be in contact with the positive electrode. The non-conductive mesh is constructed so as to allow electrons to go through the skin of the organism to the bottom plate 9. The wires 11 and 12 carry a positive charge, with wire 11 connected to top plate 8, and wire 12 connected to bottom plate 9, and provide a path for electrons to return to the system integrated within the breathing device 4.

With reference to FIG. 5, in one embodiment, the system may be powered and function using the depicted circuit design. In one embodiment, each pulse width controller comprises a microcontroller to regulate the output voltage(s) of the system.

The circuit shown comprises a rectifier bridge BR1 205, a PWM chip U1 203 and a PWM chip U3 204 (for controlling the output voltage applied for each positive electrode), and an input terminal of the rectifier bridge BR1 205 is connected to AC power, the output end is connected to the primary winding of transformer TR1 206 and the primary winding of transformer TR2 207 through filter capacitors C1 208 and C2 209. Pin 6 of PWM chip U3 204 is connected to the base of transistor Q1 210 through resistor R3 211, and the collector of transistor Q1 210 is connected to the other end of the primary winding of transformer TR1 206. Pin 6 of PWM chip U1 203 is connected to the base of transistor Q2 212 through resistor R10 213, the collector of transistor Q2 212 is connected to the other end of the primary winding of transformer TR2 207. The rectifier bridge BR1 205 and capacitors C1 208 and C2 209 rectify and filter the input AC power, providing DC (system) power for the subsequent circuit. Resistor R1 divides the above DC power and then provides operating power to the chips U1 203 and U3 204 (microcontrollers). The two chips U1 203 and U3 204 mediate the provision of pulses to the transistors Q1 210 and Q2 212, respectively to supply the switching of the transistors to drive the opening and closing of the transformers TR1 206 and TR2 207. The transformers TR1 206 and TR2 207 increase the voltage, and the increased current is AC. This AC power is respectively rectified by the rectifier bridges BR2 201 and BR4 204, and filtered by the capacitors C3 216, C4 217, C9 218, and C10 219 to obtain different higher DC voltages for output. One of the outputs (feature 110 from FIG. 4) is connected to the positive electrode top plate (feature 8 from FIG. 2) and the other output (feature 112) is connected to the second positive electrode bottom plate (feature 9 from FIG. 2). Resistors R4 221, R5 220, R11 223, and R12 222 sample the output voltages and connect to Pin 2 of U1 203 and U3 204 to provide negative feedback to chip U1 203 and U3 204 to stabilize the voltage output(s). By adjusting resistors R6, R13, or the ratio of R4 221, R5 220, or the ratio of R11 223 and R12 222, or the parameters of transformers TR1 206 and TR2 207, we can adjust the level of the two output voltages. U1 203 and U3 204 are PWM chips, for example, UC1842D8 chips (specification sheet at: https://www.datasheets360.com/part/detail/uc1842d8/-7998242350623591978/) that can provide pulses of different widths, or other similar chips can also be used. The value of R2 depends on the voltage of C2 and the operation voltage of U3 and U1, R2=Vcc/Vc2*R1. For example, if Vc2=190, Vcc=30, R1=200(1w), then R2=31.5; R7, R14: 10K; C6, C12: 0.1 uF; C5, C11: 0.0022 uf.

Correlating FIG. 4 to the embodiment of FIG. 5, the pre-stage rectification and filtering module 103 comprise a rectifier bridge BR1 205, C1, C2 where in one embodiment C2 is greater than or equal to 22 uf. The power conversion module 104 comprises TR1 and TR2. The post-stage rectification and filtering module 105 comprises BR2, C3, C4, BR4, C9, C10 wherein in one embodiment, C4 is equal to or greater than 22 uf, and C10 is greater than or equal to 22 uf. The C3, C4, C9, and C10, capacitors are configured just before the output module 106 connected to and essentially comprising the output leads and positive and negative electrodes 110, 111, 112. The over voltage protection module 109 comprises R5, R4, PIN2 OF U3, R12, R11, PIN2 OF U1, this voltage limit is set to about 10K or less volts (and could be set as high as about 80K volts, so that the necessary range of voltages can be supplied to different positive electrode configurations relative to the body of the organism). The current limiting protection module 108 comprises R6, PIN 1 OF U3, R13, PIN 1 OF U1, and in one embodiment is set to less than 10 mA, the safe level in general for the human body. The pulse width controller 107 comprises U3, PIN 6 OF U3, R3, Q1, R9, U1, PIN 6 OF U1, R10, Q2, and R8, as well as R1, R2, R7, R14, C6, C12, C5, and C11. R1 and R2 supply power to U1 and U3.

A method performed by the system according to the present disclosure is provided comprising the step of supplying a continuous flow of electrons into an air stream delivered to an organism by a breathing device, by operatively coupling a system according to the present disclosure to the breathing device. In an embodiment, the method further comprises the step of receiving electrons from the skin of the organism back into the system to thereby form an electrical circuit including the organism and system. In so doing the surrounding environment is stabilized for the organism by reducing the accumulation of negative charge, or generation of a localized, high static voltage, which can cause undesirable effects on or injury to the organism.

In one embodiment, the system is operatively coupled to the breathing device is used to support the revival of an organism, or part thereof, from a reduced metabolic state. Reduced metabolic states include organisms that are comatose, hibernating, cryogenically preserved.

In another embodiment, the system is operatively coupled to the breathing device to support a method for the recovery of an organism from a respiratory disorder. Such disorders include those where an organism is unable to uptake sufficient air or oxygen without the assistance of a breathing device (e.g. a ventilator, or mask and oxygen supply). Subjects (organisms) suffering from asthma, bronchitis, pertussis, pneumonia, COVID-19, fluid in the lungs, Emphysema, or damaged lung tissue, are all candidates for requiring assistance with breathing.

In still another embodiment, the system is operatively configured to support a method whereby electrons enter the body of the organism by way of the stomach and digestive track (e.g. in drink or food) to ameliorate conditions that can affect these body systems, such as gastroesophageal reflux (e.g. GERD or acid reflux), gall stones, Celiac Disease, Crohn's Disease, Ulcerative Colitis, Irritable Bowel Syndrome, Diverticulitis, Hemorrhoids, and anal fissures. In this exemplary method, the conductive mesh can be maintained in the conduit of an air delivery device where the air stream delivers electrons to a drink or food substance. Alternatively, the wire connecting the negative electrode can be disconnected from the negative electrode integrated into the conduit and connected to a different negative electrode comprising a conductive lining in a container holding a drink or good substance while the organism is ingesting the substance (see FIG. 8). In these embodiments, the electrons will be released to the mucosal linings and cells of the digestive track (e.g. esophagus, stomach and intestines).

As shown in FIG. 8A a flow of electrons is delivered to a container 303 holding a substance being ingested by organism via an air stream delivered through the hose 3 and conductive mesh 5 of the negative electrode connected to breathing machine 4. In FIG. 8B, the wire 2 of the system is connected to a conductive lining 305 in the container 303 to deliver the flow of electrons directly to the substance being ingested by the organism.

It will be apparent to those skilled in the art that the system and method of the present disclosure is not limited to the details of the above-mentioned exemplary embodiments, and that the present invention can be implemented in other specific forms without departing from the spirit or basic features of the present invention. Therefore, the embodiments are to be regarded as exemplary and non-limiting in every respect, and the scope of the present invention is defined by the appended claims rather than the above description, and therefore is intended to fall within the claims. All changes that are within the meaning and scope of equivalent elements are encompassed by the invention. Any reference signs in the claims should not be construed as limiting the claims involved.

Uses

Exemplary uses for the system and method of the present disclosure can be applied in conjunction with any air delivery device (such as a breathing type device) with a conduit for delivering an air stream to a substance, or to an organism, e.g. via a mask.

In the case of a breathing type device, this may include simple air oxygen delivery systems, ventilators and other specialized breathing devices. Since the supply of electrons is made before the air stream reaches the mask, there is no need to make any particular adjustments to the mask of a given breathing device. In one embodiment, the negative electrode of the system is integrated into the conduit close enough to ensure supply of a sufficient concentration of electrons/anions to the organism, but can also be far enough away from the organism to maximize safety by minimizing the inadvertent touching of the negative electrode while the system of the present disclosure is in use. By not risking having the electrode touch the face of the organism by incorporating the negative electrode into the mask (as in the prior art), the risk of exposure to a harmful voltage is minimized. So long as a concentration of electrons of not less than about 1,000 electrons per cubic centimeters can be delivered with the air stream to the nose of the organism, the placement of the conductive mesh away from the mask is adequate.

In one embodiment, the system and method of the present disclosure is used to provide a flow of electrons to, through and out of the body of an organism in need of a therapy to improve cell metabolism, healing from a disease condition, or general biological functioning. Uses according to the system and method of the present disclosure results in electrons entering into the body and providing the greatest therapeutic benefits to the part of the body where they are released from the air stream or other medium through which they may be carried into the body.

In one embodiment, when the organism is not receiving electrons through the use of a breathing device, the system may be adapted to supply a flow of electrons into a liquid that can be ingested (e.g water or other drinks) by the organism.

In another one embodiment, when the organism is not receiving electrons through the use of a breathing device, the system may be adapted to supply a flow of electrons into solid and semi-solid foods, which the organism may ingest.

Such alternative embodiments can be achieved by way of injecting an air stream that has passed through one or more layers of conductive mesh, configured as part of a conduit of an air delivery source (analogous to the air source functionality of a breathing device). The conductive mesh can be positioned at a point just prior to the conduit entering the liquid or food to be infused with electrons. In the case of infusing a drink with electrons the conductive mesh can also be placed at the opening of a bottle or container holding the liquid without the conduit being submerged in the drink liquid.

When electrons enter the body they will also circulate through the blood stream from the lungs to the rest of the body and will exit the body, released from hairs on the skin. To evaluate the response of an organism to the use of the system and methods according to the present disclosure one skilled in the art, is able, in addition to Western Medicine indicators of improved health also apply Traditional Chinese Medicine evaluation methods, whereby the health of the individual is gauged in part by how the hairs on the skin align, e.g. at the eyebrows.

In one embodiment, the system is operatively coupled to a breathing device and is used to support the revival of an organism from a reduced metabolic state. Reduced metabolic states include states in which an organism is comatose, hibernating, cryogenically preserved.

For the revival of an organism from a cryogenic or frozen like state, the system of the present disclosure can be used to support the effective resuscitation of an organism that can breathe, or part thereof that would benefit from the application of an air stream that can deliver electrons to the cells or tissue surfaces. In this regard, while the embodiments of the present disclosure refer to an air stream delivered to an organism using a breathing device, the application of the system and method of the present disclosure is equally amenable to be coupled to any delivery system of an air stream to living matter, whether or not said matter has lungs. For example, the cryopreservation and thawing of cells and tissue for reproductive technology applications, or other needs could benefit from the use of the system of the present disclosure to supply a flow of electrons during thawing. See CN109042625, CA2405404, CN105145547, US2010240127, JP2005058239, CN108130308, and KR102066218 as cryogenic methods and approaches that could be adapted for use with the present disclosure, by one skilled in the art.

In another embodiment, the system is operatively coupled to a breathing device to support the recovery of an organism from a respiratory disorder. Such disorders include those where an organism is unable to uptake sufficient air, or oxygen without the assistance of a breathing device (e.g. a ventilator, or mask and oxygen supply). Subjects (organisms) suffering from asthma, bronchitis, pertussis, pneumonia, COVID-19, fluid in the lungs, Emphysema, or damaged lung tissue, are all candidates for requiring assistance with breathing.

In still another embodiment, when electrons enter the body of the organism by way of the stomach and digestive track (in drink or food), conditions of these body systems can be ameliorated, such as gastroesophageal reflux (e.g. GERD or acid reflux), gall stones, Celiac Disease, Crohn's Disease, Ulcerative Colitis, Irritable Bowel Syndrome, Diverticulitis, Hemorrhoids, and anal fissures.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

The embodiments of the invention in which an exclusive property or privilege is claimed are defined as follows:

1. A system for supplying a flow of electrons for inhalation or ingestion by an organism, comprising:
a) an AC or DC power source for providing an input voltage to the system;
b) a pre-stage rectification and filtering module for transferring the input voltage to a power conversion module for the conversion of the input voltage into a system voltage that can power the system;
c) a post-stage rectification and filtering module for receiving a portion of the system voltage from the power conversion module and generating one or more output voltages, comprising one or more capacitors for mitigating the effect of electro-magnetic interference on the generation of the one or more output voltages and flow of electrons;
d) an output module for receiving the one or more output voltages from the post-stage rectification and filtering module, the output module comprising a negative electrode and positive electrode for, respectively, emitting the flow of electrons and receiving a flow of electrons from the skin of the organism, wherein the positive electrode has a surface area sufficient to receive said flow of electrons from substantially all of the skin of the organism;
e) an over voltage protection module connected to the output module for monitoring and limiting the amount of the one or more output voltages;
f) a current-limiting protection module connected to the output module for monitoring and limiting the amount of current running through the system; and
g) a pulse width controller connected to the output module, over voltage protection module, current limit protection module and power conversion module for controlling the one or more output voltages and flow of electrons emitted from the negative electrode;
wherein the negative electrode is integrated into a conduit of an air delivery device to supply the flow of electrons to an air stream as the air stream passes through the conduit and is delivered to the organism, or to a substance being ingested by the organism, and wherein the positive electrode is configured around the body of the organism to pull electrons emitted from substantially all of the skin of the organism back into the system.

2. The system of claim 1, wherein when the flow of electrons supplied by the negative electrode comes into contact with the air stream, anions are generated and carried by the air stream to the organism or substance being ingested by the organism.

3. The system according to claim 2, wherein the anions release electrons into the body of the organism or substance being ingested by the organism, causing electrons to move through the body of the organism.

4. The system according to claim 1, wherein the positive electrode is in contact with the skin of the organism.

5. The system according to claim 4, wherein a non-conductive woven, net-like or mesh material separates the skin of the organism from the positive electrode it is in contact with.

6. The system according to claim 1, wherein the conduit is a flexible hose.

7. The system according to claim 1, wherein the negative electrode comprises one or more layers of conductive mesh inserted into the conduit to supply the flow of electrons emitted from the negative electrode to the air stream or substance being ingested by the organism.

8. The system according to claim 7, wherein the one or more layers of conductive mesh is housed in an assembly incorporated into the conduit and configured such that the air stream passes through the one or more layers of conductive mesh to come into contact with the flow of electrons emitted from the negative electrode.

9. The system according to claim 7, wherein the one or more layers of conductive mesh form a cylinder of material with about a diameter of 50 mm and about a depth of 20 mm.

10. The system according to claim 1, wherein the positive electrode for receiving electrons emitted from the organism comprises one or more segments.

11. The system according to claim 10, wherein the one or more segments are first and second plates each with a surface area spanning the entire length and width of the organism, wherein the first plate is positioned behind or under the back of the organism and the second plate is positioned in front or over top of the organism when the system is in use.

12. The system according to claim 1, wherein a wire connects the negative electrode to the system and provides a source of electrons for the flow of electrons emitted from the negative electrode.

13. The system according to claim 12, wherein the wire can be disconnected from the negative electrode and connected to a conductive lining in a container to provide a flow of electrons to the substance being ingested by the organism.

14. A method of supplying a flow of electrons for inhalation or ingestion by an organism comprising the step of operating a system comprising:
a) an AC or DC power source for providing an input voltage to the system;
b) a pre-stage rectification and filtering module for transferring the input voltage to a power conversion module for the conversion of the input voltage into a system voltage that can power the system;
c) a post-stage rectification and filtering module for receiving a portion of the system voltage from the power conversion module and generating one or more output voltages, comprising one or more capacitors for mitigating the effect of electro-magnetic interference on the generation of the one or more output voltages and flow of electrons;
d) an output module for receiving the one or more output voltages from the post-stage rectification and filtering module, the output module comprising a negative electrode and positive electrode for, respectively, emitting the flow of electrons and receiving a flow of electrons from the skin of the organism, wherein the positive electrode has a surface area sufficient to receive said flow of electrons from substantially all of the skin of the organism;
e) an over voltage protection module connected to the output module for monitoring and limiting the amount of the one or more output voltages;
f) a current-limiting protection module connected to the output module for monitoring and limiting the amount of current running through the system; and
g) a pulse width controller connected to the output module, over voltage protection module, current limit protection module and power conversion module for controlling the one or more output voltages and flow of electrons emitted from the negative electrode;
wherein the negative electrode is integrated into a conduit of an air delivery device to supply the flow of electrons to an air stream as the air stream passes through the conduit and is delivered to the organism, or to a substance being ingested by the organism, and wherein the positive electrode is configured around the body of the organism to pull electrons emitted from substantially all of the skin of the organism back into the system.

15. The method according to claim 14, wherein the negative electrode comprises one or more layers of conductive mesh inserted into the conduit to supply the flow of electrons emitted from the negative electrode to the air stream or substance being ingested by the organism.

16. The method according to claim 14, wherein the positive electrode for receiving electrons emitted from the organism comprises one or more segments and wherein the one or more segments are first and second plates each with a surface area spanning the entire length and width of the organism, wherein the first plate is positioned behind or under the back of the organism and the second plate is positioned in front or over top of the organism when the system is in use.

17. The method of according to claim 14, wherein a wire connects the negative electrode to the system to provide a source of electrons for the flow of electrons emitted from the negative electrode and wherein the method further comprises the steps of disconnecting the wire from the negative electrode and connecting it to a conductive lining in a container to supply a flow of electrons to the substance being ingested by the organism.

18. The method according to claim 14, wherein a flow of electrons moves through the body of the organism to revive the organism from a reduced metabolic state.

19. The method according to claim 14, wherein the organism is suffering from a respiratory condition selected from asthma, bronchitis, pertussis, pneumonia, COVID-19, fluid in the lungs, Emphysema, or damaged lung tissue and the flow of electrons through the body of the organism ameliorates the condition.

20. The method according to claim 14, wherein the organism is suffering from a gastrointestinal condition selected from gastroesophageal reflux (e.g. GERD or acid reflux), gall stones, Celiac Disease, Crohn's Disease, Ulcerative Colitis, Irritable Bowel Syndrome, Diverticulitis, Hemorrhoids, and anal fissures and the flow of electrons through the organism is used to ameliorate the gastrointestinal condition.
